(19) United States
(12) Patent Application Publication
(10) Pub. No.: US 2025/0322949 A1
(43) Pub. Date: Oct. 16, 2025

(54) HEALTH MANAGEMENT SYSTEM, HEALTH MANAGEMENT METHOD, AND RECORDING MEDIUM

(71) Applicant: NEC Corporation, Tokyo (JP)

(72) Inventors: Yuki KOSAKA, Tokyo (JP); Mana HASHIMOTO, Tokyo (JP); Shuhei NOYORI, Tokyo (JP); Isao YAMADA, Tokyo (JP); Takayuki SHIBUYA, Tokyo (JP)

(73) Assignee: NEC Corporation, Tokyo (JP)

(21) Appl. No.: 19/070,646

(22) Filed: Mar. 5, 2025

(30) Foreign Application Priority Data
Apr. 16, 2024 (JP) 2024-065849

(51) Int. Cl. G16H 40/67; G06T 11/00; G06V 10/70; G06V 20/52; G06V 40/20; G16H 20/60

(52) U.S. Cl. CPC G16H 40/67; G06T 11/00; G06V 10/70; G06V 20/52; G06V 40/23; G16H 20/60; G06T 2210/04

(57) ABSTRACT

A health management system includes an image sensor installed in a house of a user, a wearable device used by the user, and a health management device capable of communicating with the image sensor and the wearable device, the health management device including at least one memory configured to store instructions; and at least one processor configured to execute the instructions to: acquire an image of an inside of the house, analyze the image to identify a room layout of the house, acquire an exercise history of the user, estimate a health condition of the user after a change in a living environment, determine an action according to the health condition after the change in the living environment, and control display of the wearable device in such a way as to display the action. It is possible to support the decision making regarding the action of the user.

This application is based upon and claims the benefit of priority from Japanese Patent Application No. 2024-065849, filed on Apr. 16, 2024, the disclosure of which is incorporated herein in its entirety by reference.

### TECHNICAL FIELD

The present disclosure relates to a health management system, a health management method, and a program.

### BACKGROUND ART

There is a case where a living environment of a user changes due to occurrence of a disaster or moving.

Reference Literature 1 (JP 2017 059261 A) describes a health information processing device that analyzes biological information, action information, and genome information and evaluates health information about a user.

### SUMMARY

An object of the present disclosure is to provide a health management device or the like that can promote health of a user after a change in a living environment.

A health management device according to an aspect of the present disclosure includes an image sensor installed in a house of a user, a wearable device used by the user, and a health management device capable of communicating with the image sensor and the wearable device, the health management device including at least one memory configured to store instructions; and at least one processor configured to execute the instructions to: acquire an image of an inside of the house imaged by the image sensor, analyze the image to identify a room layout of the house, acquire an exercise history, of the user, acquired using a sensor built in the wearable device, input the room layout of the house and the exercise history of the user to a machine learning model trained in advance to estimate a health condition of the user after a change in a living environment, determine an action according to the health condition after the change in the living environment as an action to be recommended to the user, using an action determination model that has learned a relationship between the health condition of the user and an action recommended to the user by machine learning, and control display of the wearable device in such a way as to display the determined recommended action.

A health management method according to an aspect of the present disclosure includes acquiring an image of an inside of a house imaged by an image sensor installed in the house of a user; analyzing the image to identify a room layout of the house; acquiring an exercise history, of the user, acquired using a sensor built in the wearable device used by the user; inputting the room layout of the house and the exercise history of the user to a machine learning model trained in advance to estimate a health condition of the user after a change in a living environment; determining an action according to the health condition after the change in the living environment as an action to be recommended to the user, using an action determination model that has learned a relationship between the health condition of the user and an action recommended to the user by machine learning; and controlling display of the wearable device in such a way as to display the determined recommended action.

A program according to an aspect of the present disclosure causes a computer to execute the steps of acquiring an image of an inside of a house imaged by an image sensor installed in the house of a user; analyzing the image to identify a room layout of the house; acquiring an exercise history, of the user, acquired using a sensor built in the wearable device used by the user; inputting the room layout of the house and the exercise history of the user to a machine learning model trained in advance to estimate a health condition of the user after a change in a living environment; determining an action according to the health condition after the change in the living environment as an action to be recommended to the user, using an action determination model that has learned a relationship between the health condition of the user and an action recommended to the user by machine learning; and controlling display of the wearable device in such a way as to display the determined recommended action.

The program may be stored in a non-transitory computer-readable recording medium.

### BRIEF DESCRIPTION OF THE DRAWINGS

Exemplary features and advantages of the present disclosure will become apparent from the following detailed description when taken with the accompanying drawings in which:

FIG. 1 is a block diagram illustrating an example of a configuration of a health management system including a health management device;

FIG. 2 is a block diagram illustrating an example of a configuration of the health management device;

FIG. 3 is a block diagram illustrating another example of the configuration of the health management device;

FIG. 4 is a flowchart illustrating an operation of the health management device;

FIG. 5 is a block diagram illustrating another example of the configuration of the health management device; and

FIG. 6 is a diagram illustrating an example of a hardware configuration of the health management device.

### EXAMPLE EMBODIMENT

Hereinafter, example embodiments of the present disclosure will be described in detail with reference to the drawings.

### Example Embodiment

An application example of a health management device 10 will be described. When the living environment of the user changes, the health condition of the user may also change. Therefore, the health management device 10 estimates the health condition of the user after the change in the living environment using the information about the living environment and the life log before and after the change in the living environment. Here, the user is a user of the health management device 10, and is a person whose health condition is estimated by the health management device 10. That is, the user is a person whose health can be promoted by the health management device 10. The number of users may be one or a plurality of users. The plurality of users is, for example, a family living in the same house.

An example of the change in the living environment is a change in the living environment of the user from the house in normal times to the temporary house due to the occurrence of a disaster. As a disaster occurs, a user may move to a temporary house. At this time, a change in the living environment occurs as the user moves from the house in normal times to the temporary house. The house of the user in normal times is a house before the change in the living environment, and the temporary house is a house after the change in the living environment. Here, the temporary house may be an accommodation facility such as a hotel prepared for the user. The temporary house may have a different room layout and a facility as compared with the house of the user in normal times. The temporary house and the house of the user in normal times may be different from each other in surrounding facilities and topography. Furthermore, there is a case where the community in which the user participates changes between the temporary house and the house of the user in normal times. For example, a user's lifestyle may change due to a disaster. Therefore, the health condition of the user may change before and after the change in the living environment. Therefore, the health management device 10 can promote the health of the user after the change in the living environment.

Another example of the change in the living environment is a change in the living environment due to the house of the user being disaster-stricken. When a disaster occurs, the house of the user may be damaged. However, depending on the degree of damage, the user may continue to live in the disaster-stricken house. The house in normal times is a house before the change in the living environment, and a disaster-stricken house is a house after the change in the living environment. At this time, for example, the facility of the house and the surrounding facility may be affected by the disaster. That is, the living environment may change with the disaster. Furthermore, for example, the lifestyle of the user may change due to the disaster. Therefore, the health condition of the user may change before and after the change in the living environment. Therefore, the health management device 10 can promote the health of the user after the change in the living environment.

Another example of the change in the living environment is a change in the living environment due to moving of the user. The house before the movement is a house before the change in the living environment, and the house after the movement is a house after the change in the living environment. There is a case where, for example, a room layout or the facility of a house, a surrounding facility, a location, or the like changes due to the moving. Therefore, the health condition of the user may change before and after the change in the living environment. Therefore, the health management device 10 can promote the health of the user after the change in the living environment.

Examples of the change in the living environment are not limited thereto. The change in the living environment may be a change in the working environment of the user. For example, there is a case where a user who has mainly visited the office is involved in a change to a work system that frequently works at home. There is a case where a user working at home is involved in a change to a work system that frequently visits the office. At this time, for example, the type and the amount of usage of the facility of the house of the user, and the user's lifestyle may change. Therefore, the health condition of the user may change before and after the change in the living environment. Therefore, the health management device 10 can promote the health of the user after the change in the living environment.

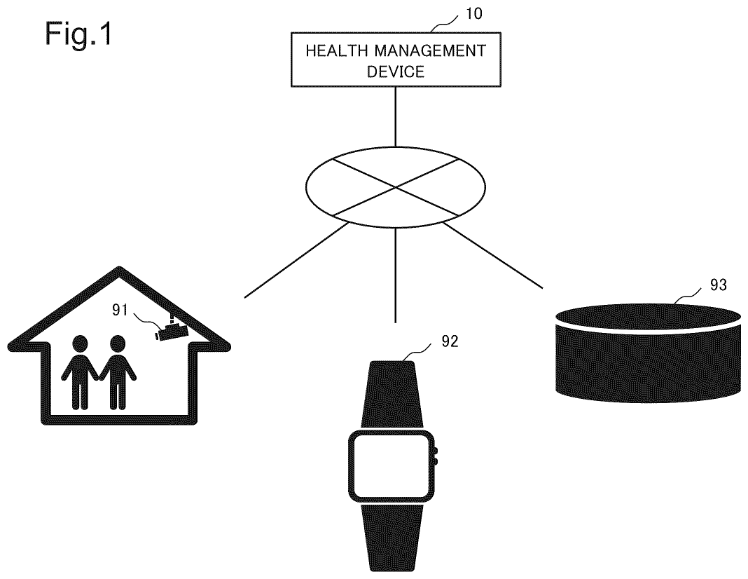

A configuration of a health management system including the health management device 10 will be described with reference to FIG. 1. FIG. 1 is a diagram illustrating an example of a configuration of a health management system including a health management device 10. Referring to FIG. 1, the health management device 10 is connected to a housing sensor 91, a communication terminal 92, and a database 93 via a wired or wireless network.

The housing sensor 91 is a sensor installed in a house. The housing sensor 91 includes a sensor installed inside the housing and a sensor installed outside the housing. The housing sensor 91 also includes a sensor included in a home appliance used in a house.

A specific example of the housing sensor 91 will be described. An example of the housing sensor 91 is an image sensor of a camera installed in a house. A user may install a camera in a house. The user may install a camera outside the house. Therefore, in a case where the user installs the camera in the house, the image sensor of the camera is included in the housing sensor 91. In FIG. 1, an image sensor of a camera installed in a house is illustrated as an example of the housing sensor 91. The number of cameras installed in the house is not limited to one. A user may install a plurality of cameras. At this time, the health management device 10 may be connected to a plurality of cameras via a wired or wireless network.

Other examples of the housing sensor 91 include a temperature sensor and a humidity sensor installed in a house. The temperature sensor is installed, for example, in a house. The temperature sensor can then measure the temperature in the house. The humidity sensor is installed, for example, in a house. The humidity sensor can then measure the humidity in the house. The temperature sensor and the humidity sensor may be installed in a home appliance, for example. For example, the temperature sensor and the humidity sensor installed in the air conditioner are examples of the housing sensor 91.

Further, another example of the housing sensor 91 is a pressure sensor. The pressure sensor is installed, for example, in a chair of a house. The pressure sensor can detect that the user is sitting on a chair. The pressure sensor may be installed on a toilet seat of a toilet of a house. The pressure sensor can detect that the user is using the toilet. The housing sensor 91 also includes a sensor included in a home appliance used in a house. Therefore, for example, when the user uses a robot vacuum cleaner, a sensor included in the robot vacuum cleaner is included in the housing sensor 91. The robotic vacuum cleaner may be provided with, for example, an infrared sensor for the purpose of mapping the house.

The housing sensor 91 is not limited thereto. The housing sensor 91 is only required to be a sensor capable of detecting the living environment of the user who lives in the housing. The number of the housing sensors 91 may be one or more. The type of the housing sensor 91 installed in the house may be one type or a plurality of types. In a case where a plurality of housing sensors 91 is installed, a plurality of housing sensors 91 of the same type may be installed, or a plurality of types of housing sensors 91 may be installed.

The communication terminal 92 is a communication terminal used by a user. An example of the communication terminal 92 is a smartphone or a wearable terminal. The communication terminal 92 may be, for example, a tablet terminal or a personal computer. FIG. 1 illustrates a wearable terminal used by a user as an example of the communication terminal 92. The number of communication terminals 92 may be one or plural. When there is a plurality of communication terminals 92, the plurality of communication terminals 92 may be a plurality of communication terminals 92 used by the same user, or may be communication terminals 92 used by a plurality of users.

A sensor is installed in a communication terminal 92. Therefore, the health management device 10 can acquire a detection result of a sensor installed in the communication terminal 92. The sensor installed in the communication terminal 92 is, for example, a global positioning system (GPS) sensor, an acceleration sensor, or a gyro sensor. The sensor installed in the communication terminal 92 is not limited thereto. In a case where the communication terminal 92 includes a camera, the image sensor of the camera is included in a sensor installed in the communication terminal 92. The user can register information using the communication terminal 92. The communication terminal 92 can transmit the registered information to the health management device 10, for example.

The database 93 stores information about the user. The information about the user includes information about a living environment and a life log of the user. That is, the database 93 is a database that stores the information about the living environment and the life log of the user. The user information stored in the database 93 is not limited thereto. The database 93 may store information about a reference person to be described later. That is, information about a person different from the user may be stored in the database 93. The information about the reference person may include, for example, information about the living environment and a life log of the reference person. The reference person will be described later. The information stored in the database 93 is not limited thereto. The database 93 may store information used by the health management device 10.

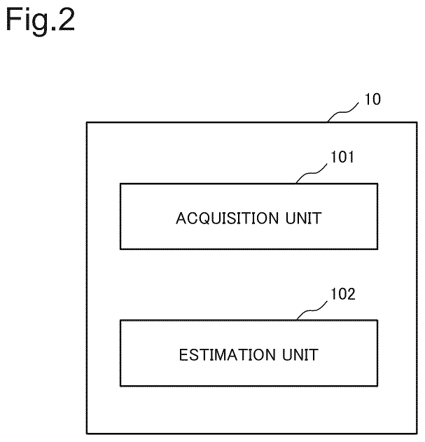

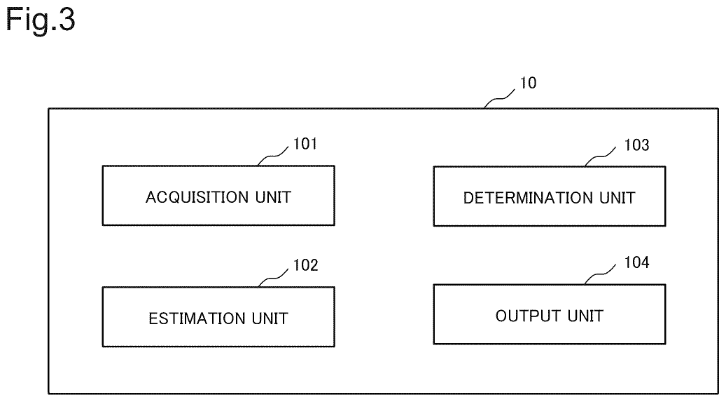

A configuration of the health management device 10 will be described with reference to FIG. 2. FIG. 2 is a block diagram illustrating an example of a configuration of the health management device 10. The health management device 10 includes an acquisition unit 101 and an estimation unit 102. The health management device 10 may include a determination unit 103 and an output unit 104. FIG. 3 is a block diagram illustrating another example of the configuration of the health management device. In the example of FIG. 3, the health management device 10 includes the acquisition unit 101, the estimation unit 102, the determination unit 103, and the output unit 104.

The acquisition unit 101 is an aspect of an acquisition means for acquiring the information about the living environment of the user and the life log of the user before and after the change in the living environment.

An example of the change in the living environment is as described above. The change in the living environment is detected by a known method. For example, a change in the living environment may be detected by registration by the user. For example, when the living environment changes, the user can register that the living environment has changed. The user may register what kind of change in the living environment has occurred. That is, the user may register the reason why the living environment has changed. Examples of the reason why the living environment has changed include moving and moving to a temporary house. The user may register the change in the living environment using the communication terminal 92, for example. The registration method by the user is not limited thereto.

The change in the living environment may be detected by, for example, a change in the information acquired by the acquisition unit 101. The acquisition unit 101 acquires the information about the living environment of the user and the life log of the user. Details of the information about the living environment of the user and the life log of the user will be described later. As the living environment changes, information about the living environment of the user or a life log of the user may change. Therefore, in a case where a predetermined change occurs in the information about the living environment of the user or the life log of the user, the living environment may change. That is, a change in the living environment may be detected. The predetermined change is a change in magnitude that occurs when the living environment changes. The predetermined change may be predetermined.

The change in the living environment may be detected, for example, based on a user's posted content on a social networking service (SNS). When the living environment changes, the user may post the change in the living environment on the SNS. Therefore, for example, a change in the living environment of the user may be detected from the word included in the posted content on the SNS. For example, in a case where the user has posted about moving, it is possible to consider a change in the living environment due to moving.

The change in the living environment may be detected, for example, based on the position information of the communication terminal 92. Based on the change in the position of the communication terminal 92, for example, a change in the house can be detected. By setting the place where the communication terminal 92 is located for a predetermined time or more as the house of the user, it is possible to detect that the place of the house of the user has changed. The predetermined time is a length of time during which the user is considered to be at his/her house, and may be determined in advance. By setting the place where the communication terminal 92 is located in a predetermined time zone as the house of the user, it is possible to detect that the place of the house of the user has changed. The predetermined time zone is a time zone in which the user is considered to be at home. The predetermined time zone is, for example, nighttime, but is not limited thereto.

The method of detecting the change in the living environment is not limited thereto. The information about the living environment of the user and the life log of the user acquired by the acquisition unit 101 before the change in the living environment is detected are the information about the living environment of the user and the life log of the user before the change in the living environment. The information about the living environment of the user and the life log of the user acquired by the acquisition unit 101 after the change in the living environment is detected are the information about the living environment of the user and the life log of the user after the change in the living environment. For example, the detection unit may detect the change in the living environment. That is, the detection unit detects a change in the living environment of the user.

The content and the acquisition method of the information about the living environment and the life log will be described.

The information about the living environment is information about the habitability of the house of the user. Depending on the living environment, the comfort of the house felt by the user varies. Therefore, the acquisition unit 101 acquires information about the habitability of the house of the user as the information about the living environment. The information about the living environment may be information about the living environment that affects the action of the user. The action of the user in life may vary depending on the living environment. Therefore, the information about the living environment may be information that affects the action of the user. Here, the information about the living environment includes information about the inside of the house of the user and information about the outside of the house of the user. Therefore, an example of the information about the living environment will be described. The information about the living environment includes at least one piece of information about the room layout of the house of the user, information about the facility of the house, information about the location of the house, and information about a community.

The information about the living environment includes information about the room layout of the house of the user. That is, the information about the room layout of the house of the user is information about the inside of the house of the user, and the information about the room layout of the house is information indicating the number and arrangement of rooms of the house. For example, in a case where the house is 1LDK, the information about the room layout includes arrangement of a living room, a living room, a dining room, a kitchen, a toilet, and a bathroom. The information of the room layout may include the direction of the house. The information of the room layout may include information of a lighting surface or the presence or absence of a window. The room layout information may include the presence or absence of stairs in the house. In a case where the house of the user is a house having a plurality of floors, the information about the room layout includes, for example, the number of floors of the house and the arrangement of rooms on each floor. In a case where the house of the user is an apartment house such as an apartment, the information about the room layout may include the floor where the house of the user is located. The room layout information is not limited thereto.

A method of acquiring information about the room layout of the house of the user by the acquisition unit 101 will be described. For example, the acquisition unit 101 can acquire information about the room layout of the house of the user by registering the user. For example, the user can register information about the room layout of the house using the communication terminal 92.

In addition, the acquisition unit 101 may acquire information about the room layout of the house from the detection result of the housing sensor 91. The acquisition unit 101 may acquire information about the room layout of the house from a detection result of a sensor included in the communication terminal 92. For example, the acquisition unit 101 acquires at least one of an image of an inside of the house imaged by a camera installed in the house and an image of the inside of the house imaged by a camera of the communication terminal 92. The acquisition unit 101 performs image analysis on at least one of the image captured by the camera installed in the house and the image captured by the camera of the communication terminal 92. As a result of the image analysis, the acquisition unit 101 can acquire information about the room layout of the house.

For example, the acquisition unit 101 can acquire information about the room layout of the house using the detection result of the infrared sensor installed in the robot vacuum cleaner. The acquisition unit 101 can acquire information about the room layout of the house by analyzing the detection result of the infrared sensor. The analysis of the detection result of the infrared sensor is performed using a known technique related to mapping of the house.

For example, the acquisition unit 101 may acquire information owned by a management company that manages a house. The acquisition unit 101 may acquire information about the room layout of the house from a server (not illustrated) of the management company. Here, the health management device 10 and the server of the management company are connected via a wired or wireless network.

The method of acquiring the information about the room layout of the house of the user by the acquisition unit 101 is not limited thereto. The acquisition unit 101 may acquire the information about the room layout of the house by combining the above-described acquisition methods. The acquisition unit 101 may store information about the room layout of the house of the user in the database 93. When the estimation unit 102 to be described later estimates the health condition, the acquisition unit 101 can read out the information about the room layout of the house of the user from the database 93. That is, the acquisition unit 101 can acquire information about the room layout of the house of the user.

The information about the living environment includes information about the facility of the house. The facility of the house includes a facility installed in the house. That is, the information about the facility of the house is information about the inside of the house of the user. The facility of the house is, for example, furniture installed in the house. Specific examples of the furniture installed in the house include a sofa, a bed, a desk, a chair, a table, a chest, and a bookshelf, but are not limited thereto. The facility of the house is, for example, a home appliance used in the house. Specific examples of home appliances used in houses include, but are not limited to, refrigerators, air conditioners, microwave ovens, televisions, cleaning robots, and dishwashers. The facility in the house may be a facility related to a lifeline including gas, water supply, electricity, and the like.

The facility of the house may include a facility provided outside the user's house. Here, the facility provided outside the house of the user refers to, for example, a facility provided in a site owned by the user. The facility provided outside the house is, for example, a parking lot for a user to park. Other examples of the facility provided outside the house include a garden and a veranda provided on the site of the user. The facility provided outside the house is not limited thereto. When the house of the user is an apartment house, the facility of the house may include a facility of a common area of the apartment house. An example of the facility of the common part is an elevator, but is not limited thereto.

The information about the facility of the house includes, for example, the type and the number of the facilities. The information about the facility of the house may include, for example, the position of the facility. In the case of the facility installed in a house, the location of the facility represents the installation location in the house. In the case of the facility provided outside the house, the location of the facility may represent a location relative to the house. Further, the information about the facility of the house may include the number of years of use of the facility.

The information about the facility of the house is not limited thereto. The information about the facility of the house may include the amount of usage of the facility. The amount of usage of each facility is, for example, a time or the number of times of using each facility in one day. For example, when the facility of the house is an air conditioner, the information about the facility of the house may include a use time and a set temperature of the air conditioner. For example, in a case where the facility of the house is a toilet, the information about the facility of the house may include the number of times of use of the toilet in one day. Further, for example, when the facility of the house is an elevator, the information about the facility of the house may include the number of times of use of the elevator in one day. The amount of usage of the facility may be represented by, for example, the usage of water or the amount of power.

A method of acquiring the information about the facility of the house of the user by the acquisition unit 101 will be described. For example, the acquisition unit 101 can acquire information about the facility of the house of the user by registering the user. For example, the user can register information about the facility of the house using the communication terminal 92.

In addition, the acquisition unit 101 may acquire information about the facility of the house from the image captured by the camera. The acquisition unit 101 includes at least one of an image of the inside of the house imaged by a camera installed inside the house, an image of the outside of the house imaged by a camera installed outside the house, and an image captured by a camera included in the communication terminal 92. The acquisition unit 101 may acquire the type and the number of facilities from the image captured by the camera. The acquisition unit 101 acquires an image of the house imaged by the camera. The acquisition unit 101 detects a facility included in the image by analyzing the image. Image analysis is performed using a known object detection technique. As a result of the analysis, the acquisition unit 101 can acquire information about the type and the number of facilities of the house.

The acquisition unit 101 may acquire the amount of usage of the facility from the image captured by the camera. Here, the image includes a moving image. The acquisition unit 101 can detect the user included in the image and detect the use of the facility by the user by the image analysis. The detection of the user and the detection of the use of the facility by the user are performed using a known image recognition technique.

The acquisition unit 101 may acquire a measurement result of a device that measures the amount of usage of the lifeline as the amount of usage of the lifeline. The device that measures the amount of usage of the lifeline is, for example, a gas meter, a water meter, or a power meter. The health management device 10 and a device for measuring the amount of usage of a lifeline are connected via a wired or wireless network. The acquisition unit 101 may acquire the amount of usage of the lifeline from a server (not illustrated) of a business operator that provides the lifeline. The health management device 10 and a server of a business operator providing a lifeline are connected via a wired or wireless network.

The acquisition unit 101 may acquire the amount of usage of the facility from the setting of each facility. The setting of each facility includes on and off of each facility. The time from on to off of the facility is the usage time of the facility. The setting of each facility may include, for example, temperature, air volume, volume, or illuminance. The setting of each facility is not limited thereto. In a case where the health management device 10 and each facility are connected via a wired or wireless network, the acquisition unit 101 may acquire the amount of usage of the facility from each facility. For example, in a case where the communication terminal 92 and each facility are connected via a wired or wireless network, the acquisition unit 101 may acquire the amount of usage of each facility from the communication terminal 92.

The method of acquiring the information about the facility of the house of the user by the acquisition unit 101 is not limited thereto. The acquisition unit 101 may acquire the information about the facility of the house by combining the above-described acquisition methods. The acquisition unit 101 may store the information about the facility of the house of the user in the database 93 in the form of a list. That is, the database 93 may store a list of residential facilities. When the estimation unit 102 to be described later estimates the health condition, the acquisition unit 101 can acquire the information about the facility of the house of the user from the list of the facilities of the house stored in the database 93.

The information about the living environment may include information about the location of the house of the user. The location of the house includes facilities around the house and topography around the house.

The facility around the house is a facility that may be used by a user. An example of the surrounding facility is a store used by a user. The store is, for example, a supermarket, a convenience store, or a drug store, but is not limited thereto. The store may be, for example, an exercise facility such as a gym. Another example of the surrounding facility is a facility related to public transportation. Examples of the facility related to public transportation include, but are not limited to, a station, a bus stop, a bus terminal, and a boarding place. Another example of the surrounding facility is a public facility. Specific examples of the public facility include, but are not limited to, a park, a hospital, a library, or a community center. The surrounding facility may include a road around the house. The road around the house includes a highway and an expressway. The facilities around the house are not limited thereto.

The information about the location of the house includes information about the surrounding facilities described above. The information about the surrounding facilities includes the type and the number of facilities. The information about the surrounding facility includes a distance from the house to the facility. Furthermore, the information about the surrounding facility may include a required time from the house to the facility. At this time, the information about the surrounding facility may include the moving means to the facility and the required time for each moving means. The information about the surrounding facility may include the use time of the facility. The information about the surrounding facilities is not limited thereto. The information about the surrounding facility may include information necessary for the user to use the facility.

The information about the location of the house includes information about the topography around the house. The information about the topography around the house is, for example, information about a height difference around the house. The momentum of the user may be different between an area with many hills and stairs and an area with many flat roads. The information about the topography around the house includes information about a height difference around the house. The height difference around the house may be represented by, for example, an altitude difference. The height difference around the house may be represented by a slope of a staircase or a slope. The information about the topography around the house is not limited thereto.

A method of acquiring information about the location of the house of the user by the acquisition unit 101 will be described. For example, the acquisition unit 101 can acquire information about the location of the house of the user by registration of the user. For example, the user may register a facility to be used by the user. The user may register a moving means to the facility. The information that can be registered by the user is not limited thereto. For example, the user can register information about the facility of the house using the communication terminal 92.

For example, the acquisition unit 101 may acquire information about the location of the house from the map information. The map information includes information about facilities and information about topography. Therefore, the acquisition unit 101 can acquire, for example, the type and the number of facilities within a range of a predetermined distance from the house. An example of the predetermined distance is a distance that the user can move. That is, the acquisition unit 101 can acquire the type and the number of facilities at the distance available to the user. The acquisition unit 101 may acquire information about the topography around the house from the map information.

The method of acquiring the information about the location of the house of the user by the acquisition unit 101 is not limited thereto. The acquisition unit 101 may acquire information about the location of the house by combining the above-described acquisition methods. The acquisition unit 101 may store the information about the surrounding facilities in the database 93 in a list format. That is, a list of surrounding facilities may be stored in the database 93. When the estimation unit 102 to be described later estimates the health condition, the acquisition unit 101 can acquire the information about the surrounding facilities of the house of the user from the list of the surrounding facilities stored in the database 93.

The information about the living environment may include information about a community. The community is, for example, an association in an area to which the user belongs. The information about the community includes participating members. The information about the community may include the purpose of participation. The user may belong to a community together with a member having a common hobby, for example. Therefore, the participation purpose of the common hobby may be included in the information about the community. The information about the community is not limited thereto. The information about the community may include information about a place where members gather to perform an activity, a participation frequency of the user, and a participation time of the user.

A method of acquiring information about a community by the acquisition unit 101 will be described. For example, the acquisition unit 101 can acquire information about a community by registering a user. For example, the user may register a participating member or a participation purpose of a community to which the user belongs. The information that can be registered by the user is not limited thereto. The user can register information about the community using the communication terminal 92, for example.

For example, the acquisition unit 101 may acquire information about the community from the position information of the communication terminal 92. A person located within a predetermined range from the communication terminal 92 of the user can be a participating member of the community. The predetermined range is a range in which the user can communicate with participating members of the community. The time during which the users and the participating members gather may be a time during which the users participate in the community. The information about the community acquired from the location information of the communication terminal 92 is not limited thereto.

The method of acquiring the information about the community by the acquisition unit 101 is not limited thereto. The acquisition unit 101 may acquire information about a community by combining the above-described acquisition methods. The acquisition unit 101 may store the information about the community in the database 93 in the form of a list. That is, a list of the community in which the user participates may be stored in the database 93. In a case where the estimation unit 102 to be described later estimates the health condition, the acquisition unit 101 can acquire information about the community of the user from the list of the community stored in the database 93.

Next, the life log will be described. The life log is information about the user's lifestyle. The life log includes at least one of the exercise history of the user and the information about the dietary life of the user.

The exercise history of the user includes daily exercise amount of the user. The exercise history may include the time of performing the exercise or the time of performing the exercise. Further, the exercise history may include an exercise content, an exercise place, and an exercise time. The exercise content is, for example, exercise such as running, walking, weight training, or cycling. The exercise content includes actions in daily life. For example, the exercise content includes moving by bicycle for shopping at a supermarket. At this time, the exercise place may be a route of reciprocating between the home and the supermarket. Furthermore, the exercise history may include, for example, movement in a house or an action for housework. The exercise history of the user is not limited thereto.

A method of acquiring the exercise history of the user by the acquisition unit 101 will be described. For example, the acquisition unit 101 can acquire the action history of the user by registering the user. The user may register the exercise content and the exercise time. As a specific example, the user may register that the user has run for 30 minutes. The information that can be registered by the user is not limited thereto. The user can register the exercise history using the communication terminal 92 including a smartphone or a wearable terminal, for example.

In addition, the acquisition unit 101 may acquire the exercise history from the image captured by the camera. Here, the image includes a moving image. The image captured by the camera includes at least one of an image of the inside of the house imaged by the camera installed inside the house, an image of the outside of the house imaged by the camera installed outside the house, and an image captured by the camera included in the communication terminal 92. The acquisition unit 101 acquires an image of the house imaged by the camera. The acquisition unit 101 detects the action of the user by analyzing the image. The acquisition unit 101 can acquire the exercise history from the detection result of the user's action. The acquisition unit 101 can acquire the exercise content and the exercise time from the action of the user imaged in the image. The acquisition unit 101 can acquire the exercise place of the user from the place of the camera that has captured the image of the user. Image analysis is performed using a known image recognition technique.

The acquisition unit 101 may acquire the exercise history from a detection result of a sensor installed in the communication terminal 92. The user may perform exercise while carrying the communication terminal 92. For example, the user may exercise by wearing the wearable terminal. The user may perform an action such as housework while carrying the smartphone. Therefore, the acquisition unit 101 can acquire the exercise history from the detection result of the sensor installed in the communication terminal 92. For example, the communication terminal 92 analyzes the detection result of the sensor and generates information about the exercise history. Therefore, the acquisition unit 101 can acquire the exercise history information from the communication terminal 92. The analysis of the detection result of the sensor installed in the communication terminal 92 may be performed by the acquisition unit 101. Analysis of the detection result of the sensor is performed using a known technique.

The method of acquiring the exercise history by the acquisition unit 101 is not limited thereto. The acquisition unit 101 may acquire the exercise history by combining the above-described acquisition methods.

The information about the dietary life of the user includes the content of the meal eaten by the user. The information regarding the dietary life may include the time or the number of times the user has had a meal. The information about dietary life may include nutritional information of a meal eaten by the user. The information about the dietary life of the user is not limited thereto.

A method of acquiring the exercise history of the user by the acquisition unit 101 will be described. For example, the acquisition unit 101 can acquire information about the user's dietary life by registering the user. For example, the user can register the taken meal using the communication terminal 92.

In addition, the acquisition unit 101 may acquire information about dietary life from an image captured by a camera. Here, the image includes a moving image. The image captured by the camera is, for example, an image captured by a camera installed in a dining room. The image captured by the camera is, for example, an image of a meal captured by the camera included in the communication terminal 92. The acquisition unit 101 acquires an image of the house imaged by the camera. The acquisition unit 101 detects the meal ingested by the user by analyzing the image. The acquisition unit 101 can acquire information about dietary life from the meal detection result. The acquisition unit 101 can acquire the time when the user has had a meal from the time when the captured image of the user is captured. Acquisition unit 101 may analyze content of a meal and acquire nutrition information of the meal.

The method of acquiring the exercise history by the acquisition unit 101 is not limited thereto. The acquisition unit 101 may acquire the exercise history by combining the above-described acquisition methods.

The life log is not limited thereto. The life log may include a vital sign of the user. Examples of vital signs are, but are not limited to, weight, blood pressure, heart rate, or pulse. The life log may include a medical history or a medication history. The life log may include the sleep history of the user. The sleep history includes sleep time, sleep quality, bedtime, and wake-up time. The acquisition unit 101 may acquire a vital sign, a medical history, a medication history, or a sleep history by registration by the user. The acquisition unit 101 may acquire a vital sign, a medical history, a medication history, or a sleep history from a detection result of a sensor installed in the communication terminal 92. Methods for acquiring a vital sign, a medical history, a medication history, or a sleep history are not limited thereto.

The method of acquiring the information about the living environment and the life log may be different depending on the acquisition time. The acquisition unit 101 acquires the information about the living environment and the life log of the user before and after the change in the user's living environment. Therefore, the acquisition unit 101 may acquire the information about the living environment and the life log by different methods before and after the change in the living environment. For example, when the change in the living environment is migration to a temporary house, the number and types of sensors installed in the house may be different between the house in normal times and the temporary house. Therefore, for example, before the change in the living environment, the acquisition unit 101 acquires 101 the information about the living environment from the housing sensor 91. On the other hand, after the change in the living environment, the acquisition unit 101 may acquire the information about the living environment by registering the user.

The acquisition unit 101 may further acquire information about the living environment and the life log of the reference person. The reference person is a person who serves as a reference person when an action is determined by the determination unit 103 described later. The reference person is, for example, a person who lives in a living environment similar to the living environment after the change of the user. By referring to the life style of a reference person who lives in a living environment similar to the living environment of the user after the change, it may be possible to recommend an effective action for the user. Here, an example of the similar living environment is that the room layout is similar to that of the house after the change in the living environment of the user. In a case where the house of the user after the change in the living environment is 1K, a person who lives in the 1K house can be a reference person. In a case where there are many hills around the house of the user after the change in the living environment, a person who lives in a house in an area with many hills can be a reference person. Here, the content of the information about the living environment and the life log are as described above. The information acquired by the acquisition unit 101 may be different between the user and the reference person.

The acquisition unit 101 can acquire the information about the living environment and the life log of the reference person by any of the above-described acquisition methods. Here, the acquisition method of the same information may be different between the user and the reference person. For example, the acquisition unit 101 acquires the exercise history of the user from a detection result of a sensor installed in the communication terminal 92. On the other hand, the acquisition unit 101 may acquire the exercise history of the reference person from the image captured by the camera.

The estimation unit 102 is an aspect of an estimation means for estimating the health condition of the user after the change in the living environment using the information about the living environment and the life log.

The health condition of the user includes a physical health condition and a mental health condition of the user. That is, the health condition indicates the physical and mental conditions of the user. Examples of the physical health condition are a risk of disorder of the musculoskeletal, a risk of worsening of a disease, or a risk of onset of a disease. The physical health condition includes being in good physical health condition. Examples of the mental health condition are mental stress, risk of developing a mental disease. The mental health condition includes being in good mental condition. Examples of the health condition are not limited thereto.

The health condition may be represented by a probability. For example, the better the health condition, the larger the number, and the worse the health condition, the smaller the number. For example, the degree of risk may be represented by probability. The health condition may be represented by ranks or scores divided into a plurality of stages. For example, mental stress may be represented by a rank divided by the intensity of stress. Furthermore, the health condition may be represented by the presence or absence of risk or stress. In addition, the health condition may be represented by a degree of change. For example, the risk of worsening of a disease may be represented by the magnitude of change in the risk of worsening. The health condition may be represented by a change rate within a specific period.

A method of estimating the health condition of the user by the estimation unit 102 will be described. The estimation unit 102 estimates the health condition of the user using the learned model. Here, a learned model used for estimating the health condition of the user is referred to as a health condition estimation model. The health condition estimation model is a model that has learned a relationship between the information about the living environment and the life log of the user, and the health condition of the user by machine learning. The estimation unit 102 uses the information about the living environment and the life log acquired by the acquisition unit 101 as inputs to the health condition estimation model. The estimation unit 102 acquires an estimation result of the health condition of the user, which is an output of the health condition estimation model. The estimation unit 102 may estimate the health condition of the user using a predetermined algorithm.

The estimation unit 102 estimates the health condition of the user after the change in the living environment using the information about the living environment and the life log before and after the change in the living environment. Using the information about the living environment and the life log before and after the change in the living environment, the estimation unit 102 can estimate the health condition after the change in the living environment based on the information before the change in the living environment.

The estimation unit 102 uses, for example, information about the room layout of the house of the user and the exercise history as inputs. At this time, the estimation unit 102 can estimate the mental stress using a health condition estimation model or a predetermined algorithm. For example, in a case where a person moves from a 3LDK house to a 1K house due to moving to a temporary house, and the daily exercise amount decreases, the person may feel mental stress due to a change in the living environment. Therefore, the estimation unit 102 can estimate the presence or absence of mental stress. The estimation unit 102 may estimate the degree of mental stress.

The estimation unit 102 uses, for example, information about the facility of the house of the user as an input. At this time, the estimation unit 102 can estimate the risk of onset of a disease or the risk of worsening of a disease using a health condition estimation model or a predetermined algorithm. The estimation unit 102 can estimate mental stress using a health condition estimation model or a predetermined algorithm. For example, when a user lives in a house damaged by a disaster, the heating facility used before the disaster may not be used after the disaster. The amount of usage of the heating facility after the disaster may decrease from the amount of usage of the heating facility before the disaster due to the restriction of power due to the disaster. As described above, the presence or absence or the amount of usage of the heating facility may change depending on the change in the living environment. At this time, it may be more difficult to manage the temperature of the house of the user than before the change in the living environment. As a result, the risk of onset of a disease or the risk of worsening of a disease may increase. In a situation where the use of the facility is restricted, the user may feel mental stress. Therefore, the estimation unit 102 can estimate the risk of onset of a disease, the risk of worsening of a disease, or the mental stress.

The estimation unit 102 uses, for example, information about the location as an input. At this time, the estimation unit 102 can estimate the risk of disorder of the musculoskeletal system or the risk of onset of a disease using a health condition estimation model or a predetermined algorithm. A case where a living environment changes due to a user's moving or moving to a temporary house will be described as an example. For example, there is a case where the house before the change in the living environment is in a region with many flat roads and there is a supermarket in a place of 5 minutes on foot, but the house after the change in the living environment is in a region with many hills and there is a supermarket only in a place of 30 minutes on foot. At this time, the user may have a case where the risk of onset of a disease is reduced by an increase in daily exercise amount. That is, the health condition may be improved. On the other hand, as the amount of exercise of the user increases, the load on the musculoskeletal system increases, and the risk of disorder of the musculoskeletal system may increase. Therefore, the estimation unit 102 estimates the risk of disorder of the musculoskeletal system or the risk of onset of a disease using a health condition estimation model or a predetermined algorithm.

The estimation unit 102 uses, for example, information about a community as an input. At this time, the estimation unit 102 can estimate the mental stress using a health condition estimation model or a predetermined algorithm. For example, due to a change in the living environment, it may be difficult to participate in a community in which the resident has participated before the change in the living environment. In a case where the user does not participate in a new community after the change in the living environment, the connection with the area is weakened, and the user may feel mental stress. Since there is no participation of the community, it is difficult to release stress, and for example, the risk of onset of a mental disease may increase. Therefore, the estimation unit 102 estimates mental stress using a health condition estimation model or a predetermined algorithm.

The estimation unit 102 may estimate the health condition of the user before the change in the living environment using the information about the living environment and the life log before the change in the living environment. The estimation unit 102 can estimate the health condition of the user before the change in the living environment using the health condition estimation model or a predetermined algorithm. The estimation unit 102 can estimate the health condition of the reference person using the information about the living environment and the life log of the reference person. The estimation unit 102 can estimate the health condition of the reference person using a health condition estimation model or a predetermined algorithm.

For the health condition estimation model or the predetermined algorithm, a known technique can be used. The health condition estimation model may be a model additionally trained for estimation of the health condition.

The health condition estimation model may be a model additionally trained for importance level estimation of the importance level used for estimation of the health condition. For example, the importance level of information used for estimation of the health condition is estimated by the additionally learned health condition estimation model. The information used for estimating the health condition is information about the living environment and a life log. The information about the living environment includes at least one piece of information about a room layout of a house, information about the facility, information about the location, and information about the community. The life log includes at least one of an exercise history, information about the dietary life, and a vital sign. The importance level of each of these pieces of information is estimated by the additionally learned health condition estimation model. The importance level is determined by repeating estimation of the health condition by the health condition estimation model.

Here, an example of a method of estimating the importance level will be described. A case where the importance level of data X is estimated will be described as an example. The data X represents some data. The data X is, for example, information of a living environment or a life log. The health condition estimated by the estimation unit 102 using the data X is compared with the health condition S′ estimated by the estimation unit 102 without using the data X. In a case where S and S′ are close values, it is estimated that the importance level of the data X for the estimation of the health condition is small. For example, an absolute error, a square error, or a cross entropy error between S and S′is used as an index of the importance level.

Another example of a method of estimating the importance level will be described. The health condition input by the user can be used to estimate the importance level of the information. The estimation unit 102 estimates the health condition of the user using the information about the living environment and the life log acquired by the acquisition unit 101. By comparing the estimated health condition with the health condition input by the user, the importance level of the information can be estimated. Here, the health condition input by the user can be acquired by the acquisition unit 101 via the communication terminal 92, for example.

The information to be acquired by the acquisition unit 101 is determined by the health condition estimation model additionally learned about the importance level estimation of the information. When the change in the living environment is, for example, a change associated with the occurrence of a disaster, the amount of power may be limited. Therefore, it may be necessary to limit the information acquired by the acquisition unit 101. Therefore, the information acquired by the acquisition unit 101 may be determined according to the importance level of the information. For example, the acquisition unit 101 acquires information with a high importance level. On the other hand, the acquisition unit 101 may not acquire information with a low importance level.

The health management device 10 may include a determination unit 103 and an output unit 104.

The determination unit 103 is an aspect of a determination means for determining an action according to the health condition after the change in the living environment as an action recommended to the user. That is, the determination unit 103 determines an action to be recommended to the user using the health condition after the change in the living environment. The action according to the health condition after the change in the living environment is an action for promoting the health of the user. For example, in a case where the health condition estimated by the estimation unit 102 is bad, the determination unit 103 may determine an action for improving the health condition of the user. In a case where the health condition estimated by the estimation unit 102 is good, the determination unit 103 may determine an action for maintaining the health condition of the user. Specific examples of the action recommended to the user include physical stretching, an action for improving the environment in the house, and exercise. The action recommended to the user may be, for example, participation in a local community.

The action recommended to the user may be an exercise using an article usable by the user. The article that can be used by the user is an article that can be used when the user performs a recommended action. An example of the article usable by the user is a stretch pole, but is not limited thereto. The information about the article usable by the user is acquired, for example, by registration of the user. The information about the article usable by the user may be acquired by image analysis from an image captured by a camera installed in the house, for example.

The action recommended to the user is not limited thereto. The action recommended to the user may be any action that can affect the health condition of the user.

The number of actions recommended to the user determined by the determination unit 103 may be one. The action recommended to the user determined by the determination unit 103 may be a recommended program including a plurality of actions. In the recommended program, for example, the order in which a plurality of actions is performed is determined. The determination unit 103 may determine at least one of the timing, the number of times, and the place at which the user performs the recommended action. The determination unit 103 may determine a period during which the user performs the recommended program. The action recommended to the user determined by the determination unit 103 may be a candidate for the action performed by the user. The determination unit 103 can determine a candidate of an action performed by the user as an action recommended to the user. The user can select an action desired to be performed from among the candidates.

The determination unit 103 determines an action to be recommended to the user using the learned model. Here, a learned model used for determining an action recommended to the user is referred to as an action determination model. The action determination model is a model that has learned a relationship between the health condition of the user and an action according to the health condition of the user by machine learning. The determination unit 103 uses the health condition after the change in the living environment of the user estimated by the estimation unit 102 as an input to the action determination model. The determination unit 103 acquires a determination result of the action recommended to the user, which is the output of the action determination model. The determination unit 103 may determine an action to be recommended to the user using a predetermined algorithm. For example, the determination unit 103 may determine an action to be recommended to the user with reference to a table in which the health condition of the user is associated with an action according to the health condition. A table in which the health condition of the user and an action according to the health condition are associated with each other is prepared in advance. The determination unit 103 may determine an action related to the health condition of the user estimated by the estimation unit 102 as an action recommended to the user.

A specific example of determination of an action recommended to the user will be described. For example, a case where the health condition that the risk of disorder of the musculoskeletal system is high is estimated by the estimation unit 102 will be described as an example. At this time, the determination unit 103 can determine the exercise effective for the joint or the muscle as the action to be recommended to the user. In a case where the estimation unit 102 estimates that the mental stress is high, the determination unit 103 may determine the stretching of the muscles related to respiration as the action recommended to the user. At this time, the determination unit 103 may determine a stretch that is considered to have an effect of alleviating anxiety and stress as an action to be recommended to the user.

In a case where the estimation unit 102 estimates that the risk of onset of a disease is high due to a decrease in the amount of exercise, the determination unit 103 may determine exercise that can be performed at home as an action to be recommended to the user. At this time, the determination unit 103 may determine a recommended program including an action for increasing the amount of exercise as an action to be recommended to the user. The recommended program may be a program in which the user continuously exercises for a predetermined period. The predetermined period is a period in which the health condition can be improved by the user continuously performing exercise. The exercise included in the recommended program may be different depending on the period. For example, the user performs exercise with a low load in the first half of the program. The user may exercise with a large load in the second half of the program. The number of times of exercise may vary depending on the period. For example, the number of exercises performed by the user may increase every time the period of the program elapses.

In a case where the plurality of risks or stresses is estimated to be high by the estimation unit 102, the determination unit 103 can determine an action to be recommended to an appropriate user by the action determination model. At this time, the score of each of the plurality of risks or stresses estimated by the estimation unit 102 is input to the action determination model. The action determination model outputs an action recommended to an appropriate user.

The determination unit 103 can determine an appropriate timing at which the user performs the recommended action as the recommendation timing. For example, the determination unit 103 may determine to periodically perform an action according to the health condition after the change in the living environment. The determination unit 103 may determine the recommendation timing according to the content of the action according to the health condition after the change in the living environment. For example, in a case where the action according to the health condition after the change in the living environment is walking, the user may obtain more effects by walking in the morning. Therefore, the determination unit 103 may determine the timing suitable for walking as morning. In a case where the action recommended to the user is a stretch that is considered to have an effect of alleviating anxiety and stress, the user may obtain a more effect by stretching before going to bed. Therefore, the determination unit 103 may determine the timing suitable for stretching as before going to bed. Examples of the timing appropriate for the user to perform the recommended action are not limited thereto. The determination unit 103 may determine the recommendation timing according to the weather. In a case where the action recommended to the user is an action performed outside the house, the determination unit 103 can determine the recommendation timing with reference to the weather information. For example, the determination unit 103 can determine a time when the weather is sunny as the recommendation timing.

The determination unit 103 can determine a place appropriate for the user to perform the recommended action as the recommended place. The determination unit 103 may determine the recommended place according to the content of the action according to the health condition after the change in the living environment. The recommended place may be determined according to the article used when the user performs the recommended action. A method of determining an appropriate place for the user to perform the recommended action is not limited thereto.

The determination unit 103 may determine an action according to the health condition after the change in the living environment and the health condition before the change in the living environment as an action to be recommended to the user. That is, the determination unit 103 determines an action to be recommended to the user using the health condition after the change in the living environment and the health condition before the change in the living environment. Using the health condition before and after the change in the living environment for determining the action recommended to the user, the determination unit 103 can determine the action according to the change in the health condition as the action recommended to the user.

A specific example of determination of an action recommended to the user according to the health condition before and after the change in the living environment will be described. A case where the estimation unit 102 estimates mental stress as the health condition and represents the degree of mental stress in 10 levels will be described as an example. Here, it is assumed that the smaller the number, the less the stress. It is assumed that the degree of mental stress before the change in the living environment is one stage, whereas the degree of mental stress after the change in the living environment is two stages. At this time, when the degree of mental stress is 2 stages, the degree of stress is small among the 10 stages. However, the stress felt by the user is increasing. Therefore, the determination unit 103 may determine a stretch that is considered to have an effect of alleviating the mental stress as the action to be recommended to the user.

Determination of the action according to the health condition before and after the change in the living environment is not limited thereto. Using the health condition before and after the change in the living environment, the determination unit 103 can determine an appropriate action according to the change in the health condition of the user.

In a case where the determination unit 103 determines a recommended action using the action determination model, the health condition after the change in the living environment and the health condition before the change in the living environment are used as inputs to the action determination model. Here, the health condition after the change in the living environment and the health condition before the change in the living environment are estimated by the estimation unit 102. The determination unit 103 acquires a determination result of the action recommended to the user, which is the output of the action determination model. The estimation unit 102 may determine an action to be recommended to the user using a predetermined algorithm.

The determination unit 103 may determine an action to be recommended to the user based on the information about the living environment and the life log of the reference person. Here, as described above, the reference person is a person in a living environment similar to the living environment after the change of the user.

The determination unit 103 can determine the reference person based on the information about the living environment of the user. That is, the determination unit 103 can determine who is referred to as a reference person when determining the action to be recommended to the user. For example, the determination unit 103 can determine a person in the living environment having a predetermined number or more of matching points with the information about the living environment of the user as the reference person. The predetermined number is set in advance. The predetermined number may be any number as long as the living environment of the user and the living environment of the reference person are considered to be similar.

For example, the determination unit 103 may determine the reference person based on the importance level of the information estimated by the health condition estimation model. The determination unit 103 may determine a person in the living environment that matches the information about the living environment estimated to have a high importance level as a reference person. The method of determining the reference person is not limited thereto. The number of reference persons may be one or plural.

When the determination unit 103 uses the action determination model, the health condition after the change in the living environment and the health condition of the reference person are used as inputs to the action determination model. Here, the health condition after the change in the living environment and the health condition of the reference person are estimated by the estimation unit 102. The determination unit 103 acquires a determination result of the action recommended to the user, which is the output of the action determination model. The estimation unit 102 may determine an action to be recommended to the user using a predetermined algorithm.

A specific example of determination of an action recommended to the user using the health condition of the reference person will be described. For example, it is assumed that the houses of the user and the reference person are both in an area with many hills, and it takes 30 minutes to walk to the supermarket. For example, the estimation unit 102 estimates the risk of disorder of the musculoskeletal system as the health condition. At this time, while the user is estimated to have a low risk of disorder of the musculoskeletal, the reference person may be estimated to have a high risk of disorder of the musculoskeletal. Since the reference person walks for 30 minutes to a supermarket in an area with many hills, it may be estimated that the risk of disorder of the musculoskeletal is high. Since the user also walks for 30 minutes to a supermarket in an area with many hills after the change in the living environment, there is a possibility that the risk of disorder of the musculoskeletal system increases. Therefore, the determination unit 103 can determine the exercise effective for the joint or the muscle as the action to be recommended to the user.

It is assumed that both the house of the user and the house of the reference person are 1K. In a case where the house of the user before the change in the living environment is, for example, 2LDK, the user may feel mental stress due to the narrowed house. However, there is a case where the degree of mental stress is estimated to be low for the reference person who lives in the same house having a 1K room layout as the user. At this time, the determination unit 103 can determine an action that affects the health condition of the reference person as an action to be recommended to the user. For example, there is a case where the number of times of use of the bathtub by the reference person is large from the information about the facility of the house of the reference person. At this time, the determination unit 103 can determine taking a bath as an action to be recommended to the user. From the exercise history of the reference person, in a case where the reference person performs running once a week, the determination unit 103 can determine that the reference person performs running once a week as the action recommended to the user.

Determination of the action recommended to the user using the health condition of the reference person is not limited thereto. By determining an action to be recommended to the user using the health condition of the reference person, the health condition of the user can be compared with the health condition of the reference person. As a result, for example, an action appropriate for the user can be determined from the difference between the health condition of the user and the health condition of the reference person. In a case where the action recommended to the user is determined using the health condition of the reference person, the determination unit 103 may further use the health condition before the change in the living environment of the user.

The output unit 104 is an aspect of an output means for outputting information about action. The output unit 104 outputs information about action according to the health condition after the change in the living environment. That is, the output unit 104 outputs information about the action recommended to the user. The information about the action recommended to the user is information necessary for the user to take an action. The information about the action recommended to the user is description of the action. For example, in a case where the information of the action recommended to the user is exercise, the description of the action may include description of part of the body to be moved, a direction of the movement, and a tip of exercise. The description of the action may include the number of times of implementing the action, the time required for the action, the name of the action, the recommendation timing, and the recommended place.

The information about the action recommended to the user is not limited thereto. In a case where the action recommended to the user is a recommended program, the information about the action recommended to the user may include an order of performing the action. The information about the action recommended to the user may include a reason for recommendation of the action. The reason for recommendation is a reason why the determination unit 103 has determined the action. The reason for recommendation may be, for example, the health condition of the user, which is used by determination unit 103 to determine the action.

The output unit 104 outputs information about the action recommended to the user in a mode that can be confirmed by the user. The output unit 104 outputs the information about the action recommended to the user in a mode that can be confirmed by the user, so that the user can obtain the information about the recommended action. The output unit 104 may output information about an action recommended to the user to the communication terminal 92. The communication terminal 92 can display information about an action recommended to the user on a screen of the communication terminal 92. The communication terminal 92 can output information about an action recommended to the user by voice from a speaker included in the communication terminal 92.

The output unit 104 may output information about an action recommended to the user to a device (not illustrated) installed in the house. The device installed in the house includes a display device. An example of the display device is a display. A display may be installed in the house. Therefore, the output unit 104 may output information about the action recommended to the user to the display. The display can display information of an action recommended to the user on the screen. The user can check the information about the action recommended to the user by viewing the display of the screen. On the screen of the display, the description of the operation can be displayed by a moving image. The device installed in the house includes a voice output device. An example of the voice output device is a speaker. The voice output device can output, by voice, information about an action recommended to the user.

The output destination by the output unit 104 is not limited thereto. The number of output destinations by the output unit 104 may be one or plural. For example, the output unit 104 may output information about an action recommended to the user to the display device and the communication terminal 92. The output destination by the output unit 104 can be determined by the determination unit 103, for example.

For example, in order to manage the health condition and action of the user, the output unit 104 may output to an output destination set in advance in such a way that a user other than the user can refer to the output destination. The user other than the user is, for example, a primary doctor or a nurse of the user.

The output destination by the output unit 104 may be different depending on the content of the action recommended to the user. When the action recommended to the user determined by the determination unit 103 is an action performed outside the house, the output unit 104 can output information about the action recommended to the user to the communication terminal 92. On the other hand, when the action recommended to the user determined by the determination unit 103 is an action performed in the house, the output unit 104 can output information about the action recommended to the user to the communication terminal 92 or a display device installed in the house.

The timing at which the output unit 104 outputs the information about the action recommended to the user may be set in advance. For example, the output unit 104 may periodically output information of an action recommended to the user. For example, the output unit 104 may output information about an action recommended to the user at 12:00 every day. The timing at which the output unit 104 outputs the information about the action recommended to the user may be set in advance by the user. The user can set a timing at which the user wants to receive information about an action recommended to the user. The output unit 104 may output information about the action recommended to the user at the timing set by the user.

The timing at which the output unit 104 outputs the information about the action recommended to the user may be determined by the content of the action. The determination unit 103 may determine a recommendation timing to perform an action recommended to the user. The output unit 104 outputs information about the action recommended to the user at the timing determined by the determination unit 103. The output unit 104 may determine a recommendation timing to perform an action recommended to the user. Therefore, the output unit 104 can output information about the action recommended to the user in accordance with the recommendation timing. For example, the output unit 104 may output information about an action recommended to the user a predetermined time before the recommendation timing. The predetermined time is, for example, a time during which the user can prepare for a recommended action. The predetermined time is a time during which the user does not forget to perform the action recommended to the user. For example, when the timing at which the output unit 104 outputs information is earlier than the recommendation timing, the user may forget to perform the recommended action. A specific example of the predetermined time is 30 minutes before, but is not limited thereto.

The timing at which the output unit 104 outputs the information about the action recommended to the user may be determined according to the recommended place of the action recommended to the user and the place where the user is. When the action recommended to the user is an action performed outside the house, the output unit 104 may output information about the action recommended to the user at a timing when the user is outside the house. In a case where the action recommended to the user is an action performed in the house, the output unit 104 may output information about the action recommended to the user at a timing when the user is in the house. Whether the user is in the house can be determined from, for example, the position information of the communication terminal 92.

The timing at which the output unit 104 outputs the information about the action recommended to the user may be determined by the determination unit 103, for example.

The timing at which the information about the action recommended to the user is output by the output unit 104 is not limited thereto. The output by the output unit 104 may be performed once or a plurality of times. In a case where the output unit 104 outputs the information about the action recommended to the user a plurality of times, the output unit 104 may output the information about the action recommended to the user to a different output destination for each output.

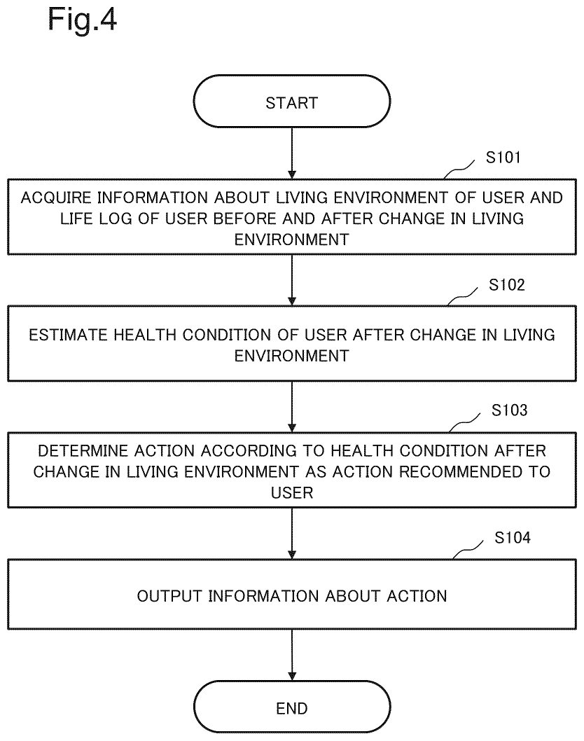

The operation of the health management device 10 including the acquisition unit 101, the estimation unit 102, the determination unit 103, and the output unit 104 will be described with reference to FIG. 4. FIG. 4 is a flowchart illustrating an operation of the health management device 10.

In step S101, the acquisition unit 101 acquires the information about the living environment of the user and the life log of the user before and after the change in the living environment. In step S102, the estimation unit 102 estimates the health condition of the user after the change in the living environment. In step S103, the determination unit 103 determines an action according to the health condition after the change in the living environment as an action to be recommended to the user. In step S104, the output unit 104 outputs information about an action recommended to the user. The health management device 10 terminates the operation.

In a case where the health management device 10 includes the acquisition unit 101 and the estimation unit 102, the health management device 10 ends the operation after steps S101 and S102.

In the present example embodiment, in the health management device 10, the acquisition unit 101 acquires the information about the living environment of the user and the life log of the user before and after the change in the living environment. The estimation unit 102 estimates the health condition of the user after the change in the living environment using the information about the living environment and the life log. For example, the living environment of the user may change due to occurrence of a disaster or moving. When the living environment changes, the health condition of the user may change. Therefore, the estimation unit 102 can manage the health condition of the user by estimate the health condition of the user after the change in the living environment. As a result, the health condition of the user can be promoted.

Here, the health condition includes at least one of a risk of disorder of a musculoskeletal, a risk of worsening of a disease, a risk of onset of a disease, mental stress, and a risk of onset of a mental disease. The health condition described above includes a health condition that may be difficult for the user to grasp. Therefore, the health condition is estimated by the estimation unit 102, so that the user can grasp his/her health condition. The health condition includes being in good physical or mental condition. At this time, the user can grasp that the health condition has not deteriorated after the change in the living environment. That is, it can be used as a guideline of life after a change in living environment. As described above, by managing the health condition, the user can easily take measures for health as necessary, for example. As a result, the health condition of the user can be promoted. It is possible to support the decision making regarding the action of the user after the change in the living environment.

The estimation unit 102 uses the information of the living environment of the user and the life log of the user acquired by the acquisition unit 101. The health condition of the user after the change in the living environment may be affected by the content of the change in the living environment. That is, the health condition of the user after the change in the living environment may be affected by how the living environment has changed. The health condition after the change in the living environment may be affected by the content of the change in the life log. That is, the health condition after the change in the living environment may be affected by how the life style of the user has changed. Therefore, the estimation unit 102 can appropriately estimate the health condition of the user after the change in the living environment using the information about the living environment of the user and the life log of the user for the estimation of the health condition.

In the present example embodiment, in the health management device 10, the determination unit 103 determines an action according to the health condition after the change in the living environment as an action to be recommended to the user. The health condition of the user may deteriorate due to a change in the living environment. In a case where the health condition estimated by the estimation unit 102 is bad, the determination unit 103 can determine an action for improving the health condition of the user. As a result, the health condition of the user may be improved. The health condition of the user may be improved by a change in the living environment. In a case where the health condition estimated by the estimation unit 102 is good, the determination unit 103 can determine an action for maintaining the health condition of the user. As a result, it may be possible to maintain or further improve the health condition of the user. That is, the determination unit 103 determines an action according to the health condition after the change in the living environment as an action recommended to the user, thereby making it possible to promote the health condition of the user after the change in the living environment.

The output unit 104 outputs information about an action recommended to the user. The output unit 104 outputs the information about the action recommended to the user, so that the user can confirm the information about the recommended action. The user can actually perform the recommended action. As a result, the user can promote his/her health.

In the health management device 10 according to the present example embodiment, the change in the living environment is a change in the living environment of the user from the house in normal times to the temporary house due to the occurrence of a disaster. As a disaster occurs, a user may move to a temporary house. There is a case where a room layout, the facility, and a location of a house change between a house in normal times and a temporary house. When the user moves to the temporary house, the community in which the user can participate may change. Furthermore, there is a case where the amount of exercise and dietary life change with a disaster. Such a change due to migration to the temporary house may put a physical or mental burden on the user. As a result, the health condition of the user may deteriorate. Therefore, the estimation unit 102 estimates the health condition of the user after the change in the living environment based on the information about the living environment and the life log before and after the change in the living environment. The determination unit 103 determines an action according to the health condition after the change in the living environment as an action to be recommended to the user, thereby making it possible to promote the health of the user.

Another example of the change in the living environment is a change in the living environment due to the house of the user being disaster-stricken. When a disaster occurs, the house of the user may be damaged. However, depending on the degree of damage, the user may continue to live in the disaster-stricken housing. At this time, for example, the facility of the house and the surrounding facility may be affected by the disaster. The user may not be able to use the facility of the house or the amount of usage of the facility may be limited. For example, the lifestyle of the user may also change due to the disaster. Under such an environment, the health condition of the user may deteriorate. Therefore, it is possible to promote the health of the user by determining an action according to the health condition after the change in the living environment as an action recommended to the user.

Another example of the change in the living environment is a change in the living environment due to moving of the user. For example, the room layout or the location of the house may be changed by the moving. The community in which the user has participated may change due to the moving. At this time, the health condition of the user may deteriorate. On the other hand, the health condition of the user may be improved due to the change in the living environment accompanying the moving. Therefore, in a case where the health condition estimated by the estimation unit 102 is poor, for example, the determination unit 103 can determine an action for improving the health condition of the user. In a case where the health condition estimated by the estimation unit 102 of the user is good, the determination unit 103 can determine, for example, an action for maintaining the health condition of the user. As a result, health of the user can be promoted.

In the health management device 10 according to the present example embodiment, the information about the living environment of the user is information about the living environment that affects the action of the user. The information about the living environment of the user includes at least one piece of information about a room layout of the house, information about a location, information about the facility, and information about a community. Using the information of the living environment that affects the action of the user, the estimation unit 102 can estimate the health condition based on the action change of the user accompanying the change in the living environment. That is, the accuracy of the estimation of the health condition by the estimation unit 102 can be improved. The determination unit 103 can determine an action to be recommended to the user using the health condition with higher accuracy. As a result, the user can promote health.

In the health management device 10 according to the present example embodiment, the life log is information about the user's lifestyle. The life log includes at least one of an exercise history and information about dietary life. Using the information about the lifestyle of the user, the estimation unit 102 can estimate the health condition based on the change in the lifestyle of the user accompanying the change in the living environment. That is, the accuracy of the estimation of the health condition by the estimation unit 102 can be improved. The determination unit 103 can determine an action to be recommended to the user using the health condition with higher accuracy. As a result, the user can promote health.

### Modifications

Modifications will be described in detail with reference to the drawings. Hereinafter, description of content overlapping with the above description will be omitted to the extent that description of the present modification is not unclear.

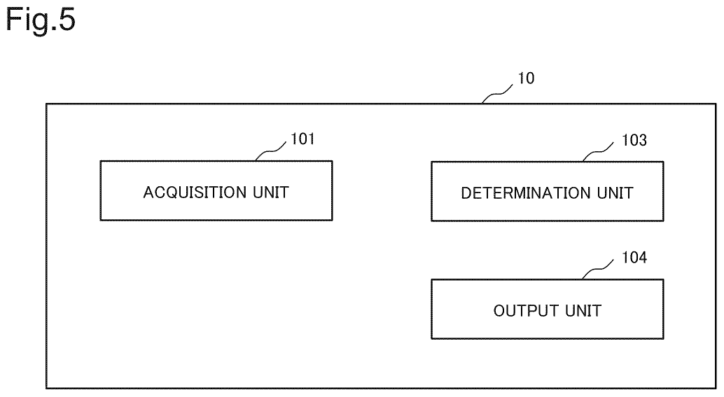

The health management device 10 may include the acquisition unit 101, the determination unit 103, and the output unit 104. FIG. 5 is a block diagram illustrating another example of the configuration of the health management device. The acquisition unit 101 and the output unit 104 are similar to those in the above example embodiment.

In a case where the health management device 10 includes the acquisition unit 101, the determination unit 103, and the output unit 104, the determination unit 103 may determine an action to be recommended to the user based on the information about the living environment of the user and the life log of the user. For example, the determination unit 103 may have the function of the estimation unit 102 described above. The health condition estimation model and the action determination model may be the same model. The method of determining the action recommended to the user based on the information about the living environment of the user and the life log of the user is not limited thereto.

In the present example embodiment, in the health management device 10, the acquisition unit 101 acquires the information about the living environment of the user and the life log of the user before and after the change in the living environment. The determination unit 103 determines an action to be recommended to the user based on the information about the living environment of the user and the life log of the user. The health condition of the user may deteriorate due to a change in the living environment. In a case where the health condition estimated by the estimation unit 102 is bad, the determination unit 103 can determine an action for improving the health condition of the user. As a result, the health condition of the user may be improved. The health condition of the user may be improved by a change in the living environment. In a case where the health condition estimated by the estimation unit 102 is good, the determination unit 103 can determine an action for maintaining the health condition of the user. As a result, it may be possible to maintain or further improve the health condition of the user. That is, the determination unit 103 determines an action according to the health condition after the change in the living environment as an action recommended to the user, thereby making it possible to promote the health condition of the user after the change in the living environment.

### Hardware Configuration Example

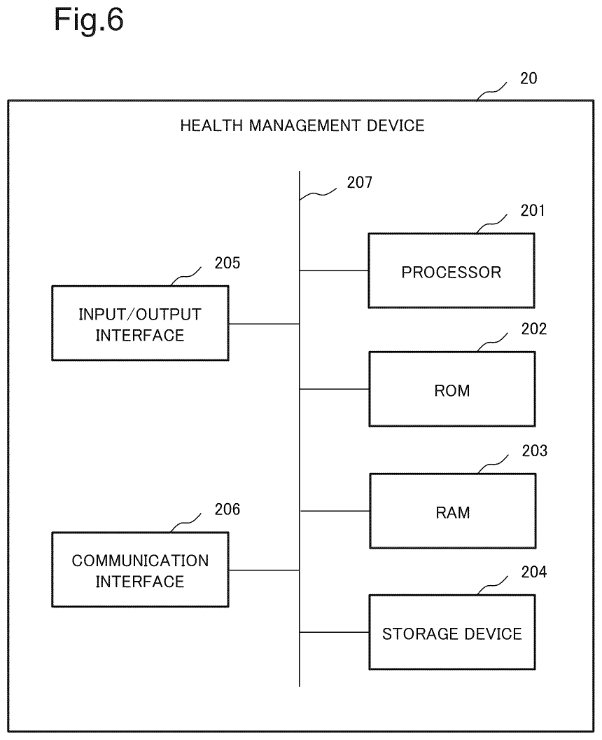

FIG. 6 is a diagram illustrating a hardware configuration example of a health management device 20 according to the present disclosure. The health management device 20 is implemented by a computer. The health management device 20 is an example of a case where the health management device 10 is implemented by a computer.

The health management device 20 includes a processor 201, a read only memory (ROM) 202, a random access memory (RAM) 203, a storage device 204 such as a hard disk for storing programs, an input/output interface 205 for inputting/outputting data, and a communication interface 206 for network connection. The components are connected via a bus 207.

The processor 201 operates an operating system to control the entire computer. Examples of the processor 201 include a central processing unit (CPU), a digital signal processor (DSP), and a graphics processing unit (GPU). The processor 201 loads a program stored in, for example, the ROM 202, the storage device 204, or the like. The processor 201 executes each process coded in the program. The processor 201 may execute processing or instructions in the illustrated flowchart based on a program.

The ROM 202 stores an application program, a program according to each example embodiment, and the like. The RAM 203 is used as a work area of the processor 201.

Examples of the storage device 204 include a semiconductor memory such as a flash memory, a hard disk drive (HDD), and the like. The storage device 204 stores, for example, an operating system (OS) program, an application program, a program according to each example embodiment, and the like.

The input/output interface 205 is connected to a peripheral device (not illustrated). The connection method may be a wired network or a wireless network.

The communication interface 206 is connected to a communication network (not illustrated) such as a local network (LAN) or a wide area network (WAN) through a wireless or wired network. The communication network may include a plurality of communication networks. As a result, the computer is connected to an external device via the communication network. The health management device 20 may have components other than those illustrated in FIG. 6. For example, the health management device 20 may include a drive device or the like. For example, the processor 201 may be mounted on a drive device or the like, and may read a program or data stored in a non-transitory tangible recording medium into the RAM 203.

When the living environment of the user changes, the health condition of the user may change.

An example of an effect of the present disclosure is to provide a health management device or the like that can promote health of a user after a change in a living environment.

While the present disclosure has been particularly shown and described with reference to each of example embodiments, the present disclosure is not limited to the above example embodiments. Various modifications that can be understood by those of ordinary skill in the art can be made to the configuration and details of the present disclosure within the scope of the present disclosure. The configurations in the example embodiments can be combined with each other without departing from the scope of the present disclosure.

Further, it is noted that the inventor's intent is to retain all equivalents of the claimed disclosure even if the claims are amended during prosecution.

Some or all of the above-described example embodiments may be described as the following Supplementary Notes, but are not limited to the following Supplementary Notes.

### (Supplementary Note 1)

A health management device including
an acquisition means for acquiring information about a living environment of a user and a life log of the user before and after a change in the living environment, and
an estimation means for estimating a health condition of the user after a change in the living environment using the information about the living environment and the life log of the user.

### (Supplementary Note 2)

The health management device according to Supplementary Note 1, further including
a determination means for determining, using an action determination model that learned a relationship between a health condition of the user and an action to be recommended to the user by machine learning, an action according to the health condition after a change in the living environment as an action to be recommended to the user

### (Supplementary Note 3)

The health management device according to Supplementary Note 2, further including
an output means for outputting information about the action.

### (Supplementary Note 4)

The health management device according to Supplementary Note 2 or 3, wherein
the estimation means estimates a health condition of a user before a change in the living environment, and
the determination means further determines an action according to the health condition before a change in the living environment as an action recommended to the user.

### (Supplementary Note 5)

The health management device according to any one of Supplementary Notes 2 to 4, wherein
the acquisition means further acquires information about a living environment and a life log of a reference person living in a living environment similar to the living environment after a change of the user, and
the determination means determines the action based on the information about the living environment and the life log of the reference person.

### (Supplementary Note 6)

A health management device including
an acquisition means for acquiring information about a living environment of a user and a life log of the user before and after a change in the living environment,
a determination means for determining an action recommended to the user using an action determination model that has learned a relationship between a health condition of the user and an action recommended to the user by machine learning based on information about a living environment of the user and a life log of the user.

### (Supplementary Note 7)

The health management device according to Supplementary Note 6, further including
an output means for outputting information about the action.

### (Supplementary Note 8)

The health management device according to Supplementary Note 6 or 7, further including
an estimation means for estimating a health condition of a user after a change in the living environment using the information about the living environment and the life log, wherein
the determination means determines an action recommended to the user based on a health condition of the user after a change in the living environment.

### (Supplementary Note 9)

The health management device according to Supplementary Note 8, wherein
the estimation means estimates the health condition of the user before a change in the living environment, and
the determination means further determines an action according to the health condition before a change in the living environment as an action recommended to the user.

### (Supplementary Note 10)

The health management device according to any one of Supplementary Notes 6 to 9, wherein
the acquisition means further acquires information about a living environment and a life log of a reference person living in a living environment similar to the living environment after a change of the user, and
the determination means determines the action based on the information about the living environment and the life log of the reference person.

### (Supplementary Note 11)

The health management device according to any one of Supplementary Notes 1 to 10, wherein
the change in the living environment is a change in a living environment of the user from a house in normal times to a temporary house due to occurrence of a disaster.

### (Supplementary Note 12)

The health management device according to any one of Supplementary Notes 1 to 11, wherein
the information about the living environment of the user is information about the living environment that affects action of the user.

### (Supplementary Note 13)

The health management device according to Supplementary Note 12, wherein
the information about the living environment of the user includes information about a room layout of a house of the user.

### (Supplementary Note 14)

The health management device according to Supplementary Note 12 or 13, wherein
the information about the living environment of the user includes information about a location of a house of the user.

### (Supplementary Note 15)

The health management device according to any one of Supplementary Notes 12 to 14, wherein
the information about the living environment of the user includes information about a facility of a house of the user.

### (Supplementary Note 16)

The health management device according to Supplementary Note 15, wherein
the information about the facility of the house of the user includes a type and the number of facilities.

### (Supplementary Note 17)

The health management device according to Supplementary Note 15 or 16, wherein
the information about the facility of the house of the user includes an amount of usage of a facility.

### (Supplementary Note 18)

The health management device according to any one of Supplementary Notes 1 to 17, wherein
the life log includes information about an exercise history of the user.

### (Supplementary Note 19)

The health management device according to any one of Supplementary Notes 1 to 18, wherein
the life log includes information about a dietary life of the user.

### (Supplementary Note 20)

The health management device according to any one of Supplementary Notes 1 to 19, wherein
the change in the living environment is a change in a living environment due to a house of the user being disaster-stricken.

### (Supplementary Note 21)

The health management device according to any one of Supplementary Notes 1 to 20, wherein
the change in the living environment is a change in a living environment due to moving of the user.

### (Supplementary Note 22)

The health management device according to any one of Supplementary Notes 1 to 21, wherein
the change in the living environment is a change in a working environment of the user.

### (Supplementary Note 23)

A health management method including
acquiring information about a living environment of a user and a life log of the user before and after a change in the living environment, and
estimating a health condition of the user after a change in the living environment using the information about the living environment and the life log of the user.

### (Supplementary Note 24)

A program for causing a computer to execute the steps of
acquiring information about a living environment of a user and a life log of the user before and after a change in the living environment, and
estimating a health condition of the user after a change in the living environment using the information about the living environment and the life log of the user.

### (Supplementary Note 25)

A recording medium storing a program for causing a computer to execute the steps of
acquiring information about a living environment of a user and a life log of the user before and after a change in the living environment, and
estimating a health condition of the user after a change in the living environment using the information about the living environment and the life log of the user.

Some or all of the configurations described in Supplementary Notes 2 to 22 dependent on the above-described Supplementary Note 1 can also be dependent on Supplementary Notes 23 to 25 by the dependency relationship similar to that of Supplementary Notes 2 to 22. Furthermore, some or all of the configurations described as the Supplementary Notes can be similarly dependent on not only the Supplementary Notes 1, and 23-25, but also various pieces of hardware and software, and various recording devices or systems for recording software without departing from the above-described example embodiments.

### Reference Signs List

10, 20 health management device
101 acquisition unit
102 estimation unit
103 determination unit
104 output unit
201 processor
202 ROM
203 RAM
204 storage device
205 input/output interface
206 communication interface
207 bus
91 housing sensor
92 communication terminal
93 database

1. A health management system comprising:
an image sensor installed in a house of a user;
a wearable device used by the user; and
a health management device capable of communicating with the image sensor and the wearable device,
the health management device comprising:
at least one memory configured to store instructions; and
at least one processor configured to execute the instructions to:
acquire an image of an inside of the house imaged by the image sensor;
analyze the image to identify a room layout of the house;
acquire an exercise history, of the user, acquired using a sensor built in the wearable device;
estimate a health condition of the user after a change in a living environment by inputting the room layout of the house and the exercise history of the user to a machine learning model trained in advance;
determine an action according to the health condition after the change in the living environment as an action to be recommended to the user, using an action determination model that has learned a relationship between the health condition of the user and an action recommended to the user by machine learning; and
control display of the wearable device in such a way as to display the action to be recommended to the user.

2. The health management system according to claim 1, wherein
the change in the living environment is a change in the living environment of the user from a house in normal times to a temporary house due to occurrence of a disaster.

3. The health management system according to claim 1, wherein the at least one processor is further configured to execute the instructions to:
estimate the health condition of the user before the change in the living environment, using the machine learning model; and
determine an action according to the health condition before the change in the living environment as the action to be recommended to the user, using the action determination model

4. The health management system according to claim 1, wherein the at least one processor is further configured to execute the instructions to:
acquire, as a reference image, an image of an inside of a house of a reference person who lives in a living environment similar to the living environment after a change of the user;
analyze the reference image to identify a room layout of the house of the reference person;
acquire an exercise history of the reference person;
estimate a health condition of the reference person, using the machine learning model; and
determine the action to be recommended to the user based on the health condition of the reference person, using the action determination model.

5. The health management system according to claim 1, wherein the at least one processor is further configured to execute the instructions to:
acquire information about a location of the house of the user;
estimate the health condition of the user after the change in the living environment by further inputting information about the location of the house of the user to the machine learning model trained in advance; and
determine an action according to the health condition after the change in the living environment as the action to be recommended to the user, using the action determination model.

6. The health management system according to claim 1, wherein the at least one processor is further configured to execute the instructions to:
acquire information about a facility of the house of the user;
estimate the health condition of the user after the change in the living environment by further inputting information about the facility of the house of the user to the machine learning model trained in advance; and
determine an action according to the health condition after the change in the living environment as the action to be recommended to the user, using the action determination model.

7. The health management system according to claim 6, wherein
the information about the facility of the house of the user includes a type and the number of facilities.

8. The health management system according to claim 6, wherein
the information about the facility of the house of the user includes an amount of usage of a facility.

9. The health management system according to claim 1, wherein the at least one processor is further configured to execute the instructions to:
acquire information about a dietary life of the user;
estimate the health condition of the user after the change in the living environment by further inputting information about the dietary life of the user to the machine learning model trained in advance to; and
determine an action according to the health condition after the change in the living environment as the action to be recommended to the user, using the action determination model.

10. The health management system according to claim 1, wherein
the change in the living environment is a change in a living environment due a house of the user being disaster-stricken.

11. The health management system according to claim 1, wherein
the change in the living environment is a change in a living environment due to moving of the user.

12. The health management system according to claim 1, wherein
the change in the living environment is a change in a working environment of the user.

13. The health management system according to claim 1, wherein the at least one processor is further configured to execute the instructions to
control a speaker installed in the house of the user in such a way as to output information about the action to be recommended to the user by voice.

14. The health management system according to claim 1, wherein the at least one processor is further configured to execute the instructions to
control display of the wearable device in such a way as to periodically display the action to be recommended to the user.

15. The health management system according to claim 1, wherein the at least one processor is further configured to execute the instructions to
control display of the wearable device in such a way that the action to be recommended to the user is displayed at a timing designated by the user.

16. The health management system according to claim 1, wherein the at least one processor is further configured to execute the instructions to:
acquire an image of the user imaged by the image sensor; and
acquire the exercise history of the user by analyzing the image of the user.

17. The action recommendation device according to claim 9, wherein the at least one processor is further configured to execute the instructions to:
acquire an image of the user imaged by the image sensor; and
acquire information about a dietary life of the user by analyzing the image of the user.

18. The health management system according to claim 1, wherein the at least one processor is further configured to execute the instructions to
control display of a display installed in a house of the user in such a way as to display the action to be recommended to the user.

19. A health management method comprising:
acquiring an image of an inside of a house imaged by an image sensor installed in the house of a user;
analyzing the image to identify a room layout of the house;
acquiring an exercise history, of the user, acquired using a sensor built in a wearable device used by the user;
estimating a health condition of the user after a change in a living environment by inputting the room layout of the house and the exercise history of the user to a machine learning model trained in advance;
determining an action according to the health condition after the change in the living environment as an action to be recommended to the user, using an action determination model that has learned a relationship between the health condition of the user and an action recommended to the user by machine learning; and
controlling display of the wearable device in such a way as to display the determined recommended action.

20. A non-transitory computer-readable recording medium that records a program for causing a computer to execute:
acquiring an image of an inside of a house imaged by an image sensor installed in the house of a user;
analyzing the image to identify a room layout of the house;
acquiring an exercise history, of the user, acquired using a sensor built in a wearable device used by the user;
estimate a health condition of the user after a change in a living environment by inputting the room layout of the house and the exercise history of the user to a machine learning model trained in advance;
determining an action according to the health condition after the change in the living environment as an action to be recommended to the user, using an action determination model that has learned a relationship between the health condition of the user and an action recommended to the user by machine learning; and
controlling display of the wearable device in such a way as to display the determined recommended action.
